(19) United States
(12) Patent Application Publication
(10) Pub. No.: US 2010/0319075 A1
(43) Pub. Date: Dec. 16, 2010

(54) STG Promoter And Its Use As A Marker For Taste And Oocyte Cells

(75) Inventors: Alexander Alexeyevich Bachmanov, Melrose Park, PA (US); Nataliya Petrovna Bosak, Philadelphia, PA (US); Yutaka Ishiwatari, Kawaski (JP)

Correspondence Address: WOODCOCK WASHBURN LLP, CIRA CENTRE, 12TH FLOOR, 2929 ARCH STREET, PHILADELPHIA, PA, 19104-2891, US

(73) Assignee: MONELL CHEMICAL SENSES CENTER, Philadelphia, PA (US)

(21) Appl. No.: 12/813,693

(22) Filed: Jun. 11, 2010

Related U.S. Application Data
(60) Provisional application No. 61/186,401, filed on Jun. 12, 2009.

(51) Int. Cl. G01N 33/48; C12N 15/63; C12N 5/10; C12Q 1/68; A01K 67/033; C12N 15/00; C12Q 1/02

(52) U.S. Cl. 800/3; 435/320.1; 435/325; 435/455; 435/6; 800/13; 800/25; 435/29; 800/21

(57) ABSTRACT

The present invention relates to a polynucleotide vector comprising the Simian taste-bud specific gene (STG) promoter operatively linked to a reporter gene. In preferred embodiments, the STG promoter is the murine ortholog of the STG promoter. In other preferred embodiments, the STG promoter is the human ortholog of the STG promoter. In some preferred embodiments, the reporter gene is green fluorescent protein (GFP). Additionally provided are vectors comprising the STG promoter operatively linked to a cre-recombinase gene.

### CROSS-REFERENCE TO RELATED APPLICATIONS

This application claims the benefit of U.S. Provisional Patent Application No. 61/186,401, filed on Jun. 12, 2009, the content of which is incorporated herein by reference in its entirety.

### TECHNICAL FIELD

The present invention relates generally to the field of cell-type specific promoters. Specifically, the present invention relates to the discovery of the mouse STG promoter and its use as a marker for taste and oocyte cells.

### BACKGROUND

The gustatory system in mammals includes taste receptor cells organized in taste buds located within gustatory papillae. Most of the taste papillae belong to three types, fungiform, foliate and vallate, and are located in the tongue. Apical ends of the taste receptor cells are exposed to the oral cavity and interact with taste stimuli, usually water-soluble chemicals. This interaction generates signals that are transmitted to the brain via afferent gustatory cranial nerves.

Taste bud cells are heterogeneous and can be classified into several subtypes based on their morphological and molecular features. There is a need to have molecular markers of taste bud cells. Several molecules have been suggested as markers of taste bud cells or their subsets. However, all the markers described in the literature have features that limit their utility. Some markers, such as certain keratins, are expressed not only in taste bud cells, but also in other tissues of the body. Some other markers, such as taste receptor or transduction molecules, are expressed only in a subset of taste bud cells.

The murine ortholog of the Simian taste bud-specific gene (STG) has the official gene symbol 2300002M23Rik and the name “RIKEN cDNA 2300002M23 gene” (murine STG gene). The gene is located on murine chromosome 17, has two exons and one intron, and encodes a protein of 349 amino acids (available through the Mouse Genome Informatics database, MGI identification number MGI:1916792) with an N-terminal signal peptide and cleavage site. The function of the protein is unknown at present. The STG gene is predominantly expressed in taste bud cells. (See Neira M, Danilova V, Hellekant G, and Azen E A, A new gene (rmSTG) specific for taste buds is found by laser capture microdissection. (2001) Mamm. Genome 12(1):60-6). It is also expressed at lower levels in a few other tissues including oocytes. The STG promoter region, however, was unknown before the present invention.

### SUMMARY

Provided herein are polynucleotide vectors comprising a species ortholog of the Simian taste-bud specific gene (STG) promoter operatively linked to a reporter gene. In preferred embodiments, the STG promoter is the murine ortholog of the STG promoter. In other preferred embodiments, the STG promoter is the human ortholog of the STG promoter. In some preferred embodiments, the reporter gene is green fluorescent protein (GFP). In other preferred embodiments, the reporter gene is glucuronidase (GUS), luciferase, chloramphenicol transacetylase (CAT) or β-galactosidase.

In preferred embodiments, the polynucleotide vector comprising the murine ortholog of the STG promoter operatively linked to a reporter gene comprises a polyadenylation signal downstream of the reporter gene.

Also provided are transformed host cells comprising a vector as described supra. In preferred embodiments, the host cell is a eukaryotic cell. In other preferred embodiments, the host cell is a human cell. In further preferred embodiments, the host cell is a taste cell. In yet further preferred embodiments, the host cell is an oocyte cell.

Also provided are methods for producing a cell that expresses a vector combining the STG promoter operatively linked to a reporter gene. The vector described supra is introduced into a cell in vitro. This cell is then cultured under conditions that allow for expression of the reporter gene. A clone is selected that expresses the reporter gene, thereby producing a cell that expresses a vector comprising the STG promoter operatively linked to the reporter gene. In preferred embodiments, the selected clone is expanded. In some preferred embodiments, the cell is an oocyte. In some embodiments, the cell is a taste tissue cell. In some preferred embodiments, the cell is a primary taste tissue cell.

Provided herein are methods for differentiating between taste cells and non-taste cells in a primary taste tissue culture. The vector described supra is introduced into cells of the primary taste tissue culture. The cells are cultured under conditions that allow for expression of the reporter gene. The reporter gene is detected, wherein cells that express the reporter gene are taste cells.

Also provided are methods for identifying a test compound that stimulates or inhibits taste cells. The vector described supra is introduced into cells of the primary taste tissue culture. The cells are cultured under conditions that allow for expression of the reporter gene. The expression of the reporter gene is detected, wherein cells that express the reporter gene are taste cells. A cell that expresses the reporter gene is exposed to the test compound, and a response of the cell that expresses the reporter gene to the test compound is detected.

Also provided are methods for identifying a test compound that stimulates or inhibits expression of a Simian taste bud-specific promoter. The vector described supra is introduced into a cell in vitro. The cell comprising the vector described supra is cultured under conditions that allow for expression of the reporter gene, thereby producing the cell that expresses a vector comprising the STG promoter operatively linked to the promoter gene. The clone that expresses the reporter gene is exposed to a test compound. The expression of the reporter gene is measured, wherein an increase in expression of the reporter gene in response to the test compound relative to the expression of the reporter gene in the absence of the test compound is indicative of a stimulant of the STG promoter and a decrease in expression of the reporter gene in response to the test compound relative to the expression of the reporter gene in the absence of the test compound is indicative of an inhibitor of the STG promoter.

Further provided are transgenic non-human mammals comprising a polynucleotide comprising the STG promoter operatively linked to a reporter gene. In preferred embodiments the transgenic non-human mammal is a mouse.

Also provided are methods for differentiating between taste cells and non-taste cells in a primary taste culture. A primary taste culture of the transgenic non-human mammal described supra is obtained. Cells of the primary taste culture are cultured under conditions that allow for expression of the reporter gene. Expression of the reporter gene is detected, wherein cells that express the reporter gene are taste cells.

Also provided are methods for analyzing a molecular, biochemical, or physiological property of a taste cell. A primary taste culture of the transgenic non-human mammal described supra is obtained. Cells of the primary taste tissue culture are cultured under conditions that allow for expression of the reporter gene. Expression of the reporter gene is detected, wherein cells that express the reporter gene are taste cells. The molecular, biochemical, or physiological property of a cell that expresses the reporter gene is then analyzed.

Provided are methods for producing cultured taste cells. A primary taste tissue culture of the transgenic non-human mammal described supra is obtained. Cells of the primary taste tissue culture are cultured under conditions that allow for expression of the reporter gene. Expression of the reporter gene is detected, wherein cells that express the reporter gene are taste cells. The cells that express the reporter gene may be selected for further culture.

Also provided are methods for identifying a test compound that stimulates or inhibits taste cells. A primary taste culture of the transgenic non-human mammal described supra is obtained. Cells of the primary taste tissue culture are cultured under conditions that allow for expression of the reporter gene. Expression of the reporter gene is detected, wherein cells that express the reporter gene are taste cells. A cell that expresses the reporter gene is exposed to the test compound, and a response of the cell that expresses the reporter gene to the test compound is detected.

Provided are methods for producing a transgenic non-human mammal comprising a polynucleotide comprising the STG promoter operatively linked to a reporter gene. A transgene is introduced into a zygote of a non-human mammal. The transgene comprises a polynucleotide comprising the STG promoter operatively linked to a reporter gene. The zygote is transplanted into a pseudopregnant mouse. The zygote is allowed to develop to term, and at least one transgenic offspring containing the transgene is identified.

Provided are methods for screening agents for their effect on the STG promoter. Cultured taste cells are selected from the transgenic non-human mammal according to the method provided supra. The taste cells are cultured under conditions for the expression of the reporter gene. An agent is added to the culture, and the expression of the reporter gene is measured.

Also provided are methods for visualizing oocytes of a mammal described herein. Oocytes are obtained from the female transgenic non-human mammal described supra. The oocytes are cultured under conditions that allow for the expression of the reporter gene, and oocytes are detected that express the reporter gene.

Further provided are methods for analyzing a molecular, biochemical or physiological property of an oocyte. Oocytes are obtained from the female transgenic non-human mammal described supra. The oocytes are cultured under conditions that allow for expression of the reporter gene. The molecular, biochemical or physiological property of an oocyte that expresses the reporter gene is then analyzed.

Further provided are methods for analyzing oocyte development and fertilization. Expression of the reporter gene in an oocyte of a female transgenic non-human mammal described supra is detected. The female transgenic non-human mammal is mated with a wild-type male non-human mammal, thereby generating a pregnant female transgenic non-human mammal. Oocyte development, fertilization and embryonic development is then analyzed in the pregnant female transgenic non-human mammal.

Further provided are methods for analyzing oocyte development and fertilization. Expression of the reporter gene in an oocyte of a female transgenic non-human mammal described supra is detected. An oocyte of the female transgenic non-human mammal expressing the reporter gene is then fertilized in vitro with sperm from a wild-type male non-human mammal, thereby generating a transgenic non-human mammal embryo. Oocyte development, and fertilization of the oocyte of the female transgenic non-human mammal is analyzed in vitro. Embryonic development of the transgenic non-human mammal is also analyzed in vitro.

Additionally provided are vectors comprising the STG promoter operatively linked to a cre-recombinase gene. In some embodiments, the cre-recombinase gene is an inducible cre-recombinase gene. In some embodiments, the cre-recombinase gene is a non-inducible cre-recombinase gene.

Also provided are transgenic non-human mammals comprising the vector comprising the STG promoter operatively linked to a cre-recombinase gene. In preferred embodiments, the transgenic non-human mammal is a mouse. In some embodiments, the transgenic non-human mammal described supra further comprises a floxed allele of another gene. In further embodiments, the cre-recombinase gene is an inducible cre-recombinase gene. In some embodiments, the cre-recombinase gene is a non-inducible cre-recombinase gene.

Provided herein are methods for generating a non-human mammal comprising a DNA deletion. A transgenic non-human mammal comprising the vector comprising the STG promoter operatively linked to a cre-recombinase gene is mated with a non-human mammal comprising a floxed allele, wherein the offspring of the mating comprise the DNA deletion.

Also provided herein are methods for generating a non-human mammal comprising a DNA deletion. A transgenic non-human mammal comprising the vector comprising the STG promoter operatively linked to a non-inducible cre-recombinase gene is mated with a non-human mammal comprising a floxed allele, wherein the offspring of the mating comprise the DNA deletion.

Also provided are methods for generating a non-human mammal comprising a DNA deletion. A transgenic non-human mammal comprising the vector comprising the STG promoter operatively linked to an inducible cre-recombinase gene is mated with a non-human mammal comprising a floxed allele, wherein the DNA deletion is induced in the offspring of the mating by the addition of the inducing ligand to the inducible cre-recombinase gene.

Also provided are non-human mammals comprising a DNA deletion generated according to any one of the methods described supra.

In preferred embodiments, methods for analyzing the effects of inactivation of the gene with the floxed allele in taste cells of the transgenic non-human mammal described supra are provided. A primary taste tissue culture of the non-human mammal is obtained. The molecular, biochemical or physiological property of a cell from the primary taste tissue culture is analyzed. The molecular, biochemical or physiological property of a cell from the primary taste culture of the non-human mammal is compared to the molecular, biochemical or physiological property of a cell from the primary taste culture of the wild-type mammal.

In further embodiments are provided methods for analyzing the effects of inactivation of the gene with the floxed allele in oocytes of the transgenic non-human mammal described supra. An oocyte of a female non-human mammal is obtained. The molecular, biochemical or physiological property of the oocyte is compared to the molecular, biochemical or physiological property of an oocyte of a wild-type mammal.

Also provided are methods for producing a vector comprising the STG promoter operatively linked to a reporter gene. The STG promoter is isolated from the genomic DNA from an animal species, and the isolated STG promoter is operatively linked to a reporter gene. In preferred embodiments, the species is a mammal. In other embodiments, the species is a mouse. In yet further embodiments, the species is a human.

Also provided are methods for identifying a species ortholog of the murine STG promoter. Sets of polymerase chain reaction (PCR) primers are designed to generate fragments of the 5′ upstream sequence of the species ortholog of the STG gene. Fragments of the 5′upstream region of the species ortholog of the STG gene are amplified using PCR utilizing genomic DNA from said species as a template and utilizing said sets of primers. In preferred embodiments, the species is a mammal. In other embodiments, the species is a human.

In further embodiments are provided methods for identifying a species ortholog of the murine STG promoter. Fragments of the 5′ upstream sequence of the species ortholog of the STG gene are isolated using restriction enzymes utilizing clones of genomic DNA fragments from said species. The isolated fragments are then sequenced. In preferred embodiments, the species is a mammal. In other embodiments, the species is a human.

### BRIEF DESCRIPTION OF THE DRAWINGS

FIG. 1 illustrates RT-PCR analysis of murine STG expression. Top: STG gene structure showing two exons, intron and location of primers used to amplify fragments shown in the lower panels. Middle: RT-PCR reactions were performed with mRNA isolated from the fungiform (FG cDNA), circumvallate (CV cDNA), foliate papilla (Fol cDNA), and brain (Brain cDNA), and with genomic DNA (gDNA). Bottom: RT-PCR reactions were performed with mRNA isolated from oocytes (oocyte cDNA) and with genomic DNA (gDNA). No signal was observed in the non-template control (NTC). See the Materials and Methods section for a list of the primers employed. The 100 by DNA Ladder (Promega, USA) was used as molecular weight markers (M).

FIG. 2 illustrates RT-PCR analysis of murine STG expression. Top: PCR reactions with murine STG gene specific primers performed on the first-strand cDNAs from the indicated mouse tissues: 1—heart, 2—brain, 3—spleen, 4—lung, 5—liver, 6—skeletal muscle, 7—kidney, 8—testis, 9—11-day embryo, 10-7-day embryo, 11—fungiform papillae, 12—circumvallate papillae, 13—gDNA (positive control), NTC—non-template (negative) control; M—molecular marker. Amplified products of the expected size (900 bp) were generated when cDNAs from circumvallate and fungiform papillae were used as templates. Bottom: Control PCR reactions with primers specific for G3PDH (a housekeeping gene) used with the same cDNAs.

FIG. 3 illustrates real-time quantitative PCR analysis of transcript abundance of the Scnn1a (ENαCα), Scnn1b (ENaCβ), Scnn1g (ENaCγ), and STG genes in fungiform papillae of 129P3/J (129) (left panel) and C57BL/6J (B6) (right panel) mice.

FIG. 4 illustrates the restriction map of the 5′ upstream region of the murine STG gene.

FIG. 5 illustrates PCR amplification of fragments of the 5′ upstream promoter region of the murine STG gene and cloning into the expression vector pAcGFP1-1. Several fragments were amplified. The largest fragment spans a whole ˜5.5 kb intergenic interval between the murine STG gene and the predicted gene, 674169 (LOC674169). The smallest (˜1.5 kb) fragment was used to construct a GFP expression vector for an experiment shown in FIG. 6. Molecular weight markers are indicated in the right columns of the gel images.

FIG. 6 illustrates the analysis of STG-induced GFP expression in mouse oocytes. Top: Oocytes injected with a GFP reporter construct containing 1,179 by fragment of genomic DNA 5′ upstream of the murine STG gene. Bottom: Oocytes injected with a GFP reporter construct without STG promoter sequences (negative control). Left: ultraviolet (UV) light; right: bright light.

### DETAILED DESCRIPTION OF ILLUSTRATIVE EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

All references cited herein are incorporated by reference in their entirety and for all purposes.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “STG” as used herein describes the Simian taste-bud specific gene.

The term “murine STG” as used herein describes the murine ortholog of the Simian taste-bud specific gene.

The term “human STG” as used herein describes the human ortholog of the Simian taste-bud specific gene.

The term “ENaCα” as used herein describes the sodium channel, nonvoltage-gated 1, alpha gene, also known as ENaCA or Scnn1a.

The term “ENaCβ” as used herein describes the sodium channel, nonvoltage-gated 1, beta gene, also known as ENaCB or Scnn1b.

The term “ENaCγ” as used herein describes the sodium channel, nonvoltage-gated 1, gamma gene, also known as ENaCG or Scnn1g.

The term “promoter” as used herein describes minimal sequence sufficient to direct transcription. Also included in the definition are those promoter elements which are sufficient to render promoter-dependent gene expression controllable for cell-type specific, tissue-specific or inducible by external signals or agents; such elements may be located in the 5′ or 3′ regions of the native gene.

The term “operably linked” as used herein describes when a gene and one or more regulatory sequences are connected in such a way as to permit gene expression when the appropriate molecules (e.g., transcriptional activator proteins) are bound to the regulatory sequences.

The term “reporter gene” as used herein describes a gene whose expression may be assayed. Such genes include, without limitation, green fluorescent protein (GFP), glucuronidase (GUS), luciferase, chloramphenicol transacetylase (CAT), and β-galactosidase.

The term “transformed cell” as used herein describes a cell into which (or into an ancestor of which) has been introduced, by means of recombinant DNA techniques, a DNA molecule.

The term “transgene” as used herein describes any piece of DNA which is inserted by artifice into a cell, and becomes part of the genome of the organism which develops from that cell. Such a transgene may include a gene that is partly or entirely heterologous (i.e. foreign) to the transgenic organism, or may represent a gene homologous to an endogenous gene of that organism.

The term “transgenic” as used herein describes any cell or organism which includes a DNA sequence which is inserted by artifice into the cell or into an organism that develops from such a cell. As used herein, the transgenic organisms are generally transgenic mammals, including rodents, such as mice, and the DNA is inserted by artifice into the nuclear genome.

The term “transformation” as used herein describes any method for introducing foreign molecules into cells. Examples of transformation include, without limitation, lipofection, calcium phosphate precipitation, retroviral delivery, electroporation and biolistic transformation.

The term “cre-recombinase” as used herein describes a site-specific DNA recombinase that can catalyze the recombination of DNA between loxP sequences. Cre-recombinase cleaves the double stranded DNA at both loxP sites, and the strands are then rejoined by DNA ligase. The Cre/loxP system was initially discovered in the filamentous P1 phage and has become a valuable tool for the induction of site-specific DNA recombination. A variety of cre-recombinases are available. Depending on the position and orientation of loxP sites, gene deletions, duplications, inversions, or chromosomal translocations may be generated. Inducible forms of cre-recombinase have been developed. One example comprises cre-recombinase fused to the ligand-binding domain of a mutated human estrogen receptor that recognizes tamoxifen or 4-hydroxytamoxifen. This ligand inducible cre-estrogen receptor fusion protein is retained in the cytoplasm and is translocated to the nucleus upon addition of tamoxifen or 4-hydroxytamoxifen.

The term “floxed allele” as used herein describes an allele flanked by a direct repeat of loxP sites. In the presence of Cre-recombinase, the double stranded DNA is cleaved at both loxP sites, and the strands are then rejoined by DNA ligase, leading to a deletion of the allele that was flanked by loxP sites.

It is to be understood that the embodiments described herein are not limited to particular methods, reagents, compounds, compositions or biological systems, which can, of course, vary. It is also to be understood that the terminology used herein is for the purpose of describing particular promoter or reporter gene sequences only, and is not intended to be limiting.

Provided is a polynucleotide vector comprising a species ortholog of the Simian taste-bud specific gene (STG) promoter operatively linked to a reporter gene. In preferred embodiments, the STG promoter is the murine ortholog of the STG promoter. In other preferred embodiments, the STG promoter is the human ortholog of the STG promoter. The sequence of the human ortholog of the murine STG gene (C6orf15: chromosome 6 open reading frame 15) is available from GenBank, Accession No: NM_{—}014070. In some preferred embodiments, the reporter gene is green fluorescent protein (GFP). In other preferred embodiments, the reporter gene is glucuronidase (GUS), luciferase, chloramphenicol transacetylase (CAT) or β-galactosidase. The vector may include one or more additional regulatory elements. Additional regulatory elements may include sequences encoding suitable mRNA ribosomal binding sites, and sequences which control the termination of transcription and translation. Mammalian expression vectors may also comprise nontranscribed elements such as an origin of replication, a suitable promoter and enhancer linked to the gene to be expressed, other 5′ or 3′ flanking nontranscribed sequences, 5′ or 3′ nontranslated sequences such as necessary ribosome binding sites, splice donor and acceptor sites, and transcriptional termination sequences. An origin of replication that confers the ability to replicate in a host, and a selectable gene to facilitate recognition of transformants, may also be incorporated. In addition, the expression vector consists of a positive selectable marker that allows for selection of recipient hosts that have taken up the expression vector.

In preferred embodiments, the polynucleotide vector comprising the murine ortholog of the STG promoter operatively linked to a reporter gene comprises a polyadenylation signal downstream of the reporter gene.

Also provided are transformed host cells comprising a vector as described supra. Transformed host cells are cells which have been transfected with expression vectors generated by recombinant DNA techniques. Various cell culture systems can be employed, including primary taste tissue cultures or primary oocyte cultures.

In preferred embodiments, the host cell is a eukaryotic cell. In some preferred embodiments, the host cell is a mammalian cell. In other preferred embodiments, the host cell is a human cell.

In further preferred embodiments, the host cell is a taste cell.

In yet further preferred embodiments, the host cell is an oocyte cell.

Also provided are methods for producing a cell that expresses a vector combining the STG promoter operatively linked to a reporter gene. The vector described supra is introduced into a cell in vitro. Examples of transfection methods that may be employed include liposome-mediated (e.g., using Transfast or Lipofectamine) or adenoviral transfection systems or methods. The transfected cell is then cultured under conditions that allow for expression of the reporter gene. A clone is selected that expresses the reporter gene, thereby producing a cell that expresses a vector comprising the STG promoter operatively linked to the reporter gene. In preferred embodiments, the selected clone is expanded. In some preferred embodiments, the cell is an oocyte. In some embodiments, the cell is a taste tissue cell. In some embodiments, the cell is a primary taste tissue cell.

Provided herein are methods for differentiating between taste cells and non-taste cells in a primary taste tissue culture. The vector described supra is introduced into cells of the primary taste tissue culture. The cells are cultured under conditions that allow for expression of the reporter gene. The reporter gene is detected, wherein cells that express the reporter gene are taste cells.

Provided are methods for identifying a test compound that stimulates or inhibits taste cells. The vector described supra is introduced into cells of the primary taste tissue culture. The cells are cultured under conditions that allow for expression of the reporter gene. The expression of the reporter gene is detected, wherein cells that express the reporter gene are taste cells. A cell that expresses the reporter gene is exposed to the test compound, and a response of the cell that expresses the reporter gene to the test compound is detected.

Further provided are methods for identifying a test compound that stimulates or inhibits expression of a STG promoter. The vector described supra is introduced into a cell in vitro. The cell comprising the vector described supra is cultured under conditions that allow for expression of the reporter gene, thereby producing the cell that expresses a vector comprising the STG promoter operatively linked to the promoter gene. The clone that expresses the reporter gene is exposed to a test compound. The expression of the reporter gene is measured, wherein an increase in expression of the reporter gene in response to the test compound relative to the expression of the reporter gene in the absence of the test compound is indicative of a stimulant of the STG promoter and a decrease in expression of the reporter gene in response to the test compound relative to the expression of the reporter gene in the absence of the test compound is indicative of an inhibitor of the STG promoter.

Further provided are transgenic non-human mammals comprising a polynucleotide comprising the STG promoter operatively linked to a reporter gene. In preferred embodiments the transgenic non-human mammal is a mouse.

Also provided are methods for differentiating between taste cells and non-taste cells in a primary taste culture. A primary taste culture of the transgenic non-human mammal described supra is obtained. Cells of the primary taste culture are cultured under conditions that allow for expression of the reporter gene. Expression of the reporter gene is detected, wherein cells that express the reporter gene are taste cells.

Also provided are methods for analyzing a molecular, biochemical, or physiological property of a taste cell. A primary taste culture of the transgenic non-human mammal described supra is obtained. Cells of the primary taste tissue culture are cultured under conditions that allow for expression of the reporter gene. Expression of the reporter gene is detected, wherein cells that express the reporter gene are taste cells. The molecular, biochemical, or physiological property of a cell that expresses the reporter gene is then analyzed.

Provided are methods for producing cultured taste cells. A primary taste tissue culture of the transgenic non-human mammal described supra is obtained. Cells of the primary taste tissue culture are cultured under conditions that allow for expression of the reporter gene. Expression of the reporter gene is detected, wherein cells that express the reporter gene are taste cells. The cells that express the reporter gene may be selected for further culture.

Also provided are methods for identifying a test compound that stimulates or inhibits taste cells. A primary taste culture of the transgenic non-human mammal described supra is obtained. Cells of the primary taste tissue culture are cultured under conditions that allow for expression of the reporter gene. Expression of the reporter gene is detected, wherein cells that express the reporter gene are taste cells. A cell that expresses the reporter gene is exposed to the test compound, and a response of the cell that expresses the reporter gene to the test compound is detected.

Several techniques may be used to measure the responses of taste cells to exposure to the test compound including, for example: electrophysiological recordings of activity, or imaging with Ca²⁺, Na⁺ or voltage sensitive assays. For example, changes in intracellular Ca²⁺ levels may be monitored by the fluorescence of indicator dyes such as indo or fura. Another technique that may be used to measure the responses of taste cells to exposure to the test compound is the measurement of changes in cAMP, cGMP, IP₃, and DAG levels. The control to which the response of taste cells to the test compound may be compared may be the level of cell activity before exposure to the test compound, or the level of activity of cells that are not exposed to the test compound.

Provided are methods for producing a transgenic non-human mammal comprising a polynucleotide comprising the STG promoter operatively linked to a reporter gene. A transgene is introduced into a zygote of a non-human mammal. The transgene comprises a polynucleotide comprising the STG promoter operatively linked to a reporter gene. The zygote is transplanted into a pseudopregnant mouse. The zygote is allowed to develop to term, and at least one transgenic offspring containing the transgene is identified.

Provided are methods for screening agents for their effect on the STG promoter. Cultured taste cells are selected from the transgenic non-human mammal according to the method provided supra. The taste cells are cultured under conditions for the expression of the reporter gene. An agent is added to the culture, and the expression of the reporter gene is measured.

Also provided are methods for visualizing oocytes of a mammal described herein. Oocytes are obtained from the female transgenic non-human mammal described supra. The oocytes are cultured under conditions that allow for the expression of the reporter gene, and oocytes are detected that express the reporter gene.

Further provided are methods for analyzing a molecular, biochemical or physiological property of an oocyte. Oocytes are obtained from the female transgenic non-human mammal described supra. The oocytes are cultured under conditions that allow for expression of the reporter gene. The molecular, biochemical or physiological property of an oocyte that expresses the reporter gene is then analyzed.

Further provided are methods for analyzing oocyte development and fertilization. Expression of the reporter gene in an oocyte of a female transgenic non-human mammal described supra is detected. The female transgenic non-human mammal is mated with a wild-type male non-human mammal, thereby generating a pregnant female transgenic non-human mammal. Oocyte development and fertilization are then analyzed in the pregnant female transgenic non-human mammal.

Further provided are methods for analyzing oocyte development and fertilization. Expression of the reporter gene in an oocyte of a female transgenic non-human mammal described supra is detected. An oocyte of the female transgenic non-human mammal expressing the reporter gene is then fertilized in vitro with sperm from a wild-type male non-human mammal, thereby generating a transgenic non-human mammal embryo. Oocyte development, and fertilization of the oocyte of the female transgenic non-human mammal is analyzed in vitro. Embryonic development of the transgenic non-human mammal is also analyzed in vitro.

Additionally provided are vectors comprising the STG promoter operatively linked to a cre-recombinase gene. In some embodiments, the cre-recombinase gene is an inducible cre-recombinase gene. In some embodiments, the cre-recombinase gene is a non-inducible cre-recombinase gene.

Also provided are transgenic non-human mammals comprising the vector comprising the STG promoter operatively linked to a cre-recombinase gene. In preferred embodiments, the transgenic non-human mammal is a mouse. In some embodiments, the transgenic non-human mammal described supra further comprises a floxed allele of another gene. In some embodiments, the cre-recombinase gene is an inducible cre-recombinase gene. In some embodiments, the cre-recombinase gene is a non-inducible cre-recombinase gene.

Provided herein are methods for generating a non-human mammal comprising a DNA deletion. A transgenic non-human mammal comprising the vector comprising the STG promoter operatively linked to a cre-recombinase gene is mated with a non-human mammal comprising a floxed allele, wherein the offspring of the mating comprise the DNA deletion.

Also provided herein are methods for generating a non-human mammal comprising a DNA deletion. A transgenic non-human mammal comprising the vector comprising the STG promoter operatively linked to a non-inducible cre-recombinase gene is mated with a non-human mammal comprising a floxed allele, wherein the offspring of the mating comprise the DNA deletion.

Also provided are methods for generating a non-human mammal comprising a DNA deletion. A transgenic non-human mammal comprising the vector comprising the STG promoter operatively linked to an inducible cre-recombinase gene is mated with a non-human mammal comprising a floxed allele, wherein the DNA deletion is induced in the offspring of the mating by the addition of the ligand to the inducible cre-recombinase.

Also provided are non-human mammals comprising a DNA deletion generated according to any one of the methods described supra.

In preferred embodiments, methods for analyzing the effects of inactivation of the gene with the floxed allele in taste cells of the transgenic non-human mammal described supra are provided. A primary taste tissue culture of the non-human mammal is obtained. The molecular, biochemical or physiological property of a cell from the primary taste tissue culture is analyzed. The molecular, biochemical or physiological property of a cell from the primary taste culture of the non-human mammal is compared to the molecular, biochemical or physiological property of a cell from the primary taste culture of the wild-type mammal.

In further embodiments are provided methods for analyzing the effects of inactivation of the gene with the floxed allele in oocytes of the transgenic non-human mammal described supra. An oocyte of a female non-human mammal is obtained. The molecular, biochemical or physiological property of the oocyte is compared to the molecular, biochemical or physiological property of an oocyte of a wild-type mammal.

Also provided are methods for producing a vector comprising the STG promoter operatively linked to a reporter gene. The STG promoter is isolated from the genomic DNA from an animal species, and the isolated STG promoter is operatively linked to a reporter gene. In preferred embodiments, the species is a mammal. In other embodiments, the species is a mouse. In yet further embodiments, the species is a human.

Also provided are methods for identifying a species ortholog of the murine STG promoter. Sets of polymerase chain reaction (PCR) primers are designed to generate fragments of the 5′ upstream sequence of the species ortholog of the STG gene. Fragments of the 5′ upstream region of the species ortholog of the STG gene are amplified using PCR utilizing genomic DNA from said species as a template and utilizing said sets of primers. The amplified fragments are then sequenced. In preferred embodiments, the species is a mammal. In other embodiments, the species is a human.

In further embodiments are provided methods for identifying a species ortholog of the murine STG promoter. Fragments of the 5′ upstream sequence of the species ortholog of the STG gene are isolated using restriction enzymes utilizing clones of genomic DNA fragments from said species. The isolated fragments are then sequenced. In preferred embodiments, the species is a mammal. In other embodiments, the species is a human.

### EXAMPLES

### Example 1

### Materials and Methods

Collection of Taste Tissues.

Tongues were excised from euthanized C57BL/6J and 129P3/J mice and placed in the calcium-free Tyrode's buffer. The excised tongue was injected subepithelially with a mixture of dispase II (2 mg/ml) and collagenase A (1 mg/ml) in Ca²⁺-free Tyrode's solution and incubated at 37° C. for 20 min. The epithelium was peeled off and washed three times with calcium-containing PBS. The circumvallate and foliate papillae were excided, and the anterior part of the lingual epithelium with the highest density of fungiform papillae was also collected.

RNA Extraction.

Total RNA was extracted using the Absolutely RNA Microprep Kit (Stratagene, La Jolla, Calif.) including a DNase step to reduce genomic DNA to undetectable levels. In addition, 230 μM sodium acetate (pH 5.2) and 40 μg glycogen (Roche Applied Science, Indianapolis, Ind.) as a carrier was added prior to precipitation with ice-cold ethanol.

Reverse Transcription PCR.

First-strand complimentary DNA was synthesized by priming with random hexamers using Superscript II reverse transcriptase (Invitrogen, Carlsbad, Calif.) following the manufacture's instructions. PCR analyses was performed with these samples or purchased cDNA (Mouse MTC Panel I and II; Clontech) using gene-specific primers (5′-TTCCTTCACCAGCCTCCTTA-3′ (SEQ ID NO: 1); 5′-GCTGCAAACCAGTTGTGAGA-3′ (SEQ ID NO: 2)) and G3PDH to monitor (exclude) possible contamination from genomic DNA.

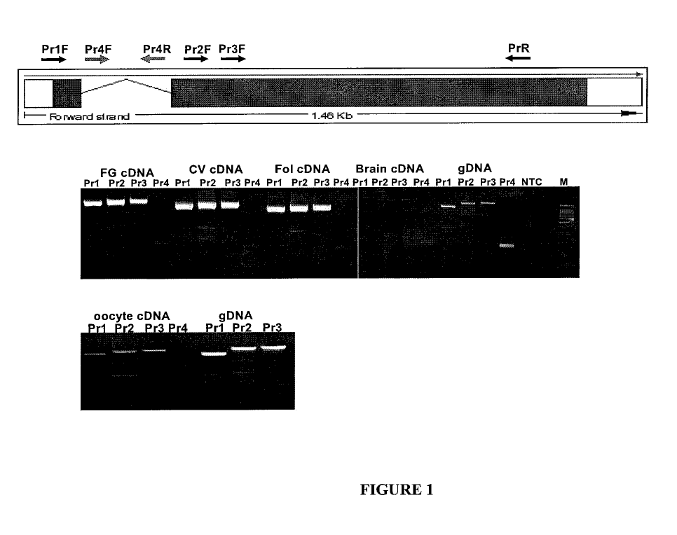

STG-specific primers (See FIG. 1):

| | | |
|---|---|---|
| PrR: | GCTGCAAACCAGTTGTGAGA | (SEQ ID NO: 3) |
| Pr1F: | TTCCTTCACCAGCCTCCTTA | (SEQ ID NO: 4) |
| Pr2F: | TGCAGAGCCACGCAGGTGGGAG | (SEQ ID NO: 5) |
| Pr3F: | CGACCAACAGGATAAAGGCTG | (SEQ ID NO: 6) |
| Pr4F: | GTACTGGGGTCCTTGGGAAC | (SEQ ID NO: 7) |
| Pr4R: | CCCGCTACTCTTTTGACTCG | (SEQ ID NO: 8) |

Quantitative Real-Time PCR.

Total RNA was extracted using Absolutely RNA Microprep Kit (Stratagene, La Jolla, Calif.) from peeled-off epithelial pieces that contained foliate, circumvallate, and fungiform taste buds. Approximately equal amounts of total RNA from these tissues were reverse transcribed into cDNA using Superscript II reverse transcriptase (Invitrogen, Carlsbad, Calif.). The quantitative PCR (qPCR) reactions were performed using the 7300 Real Time PCR System (PE Applied Biosystems, Foster City, Calif.) with TaqMan PCR Master reagents and ABI TaqMan Assay-on-Demand probe/primer sets Mm01182998_g1 (Scnn1a), Mm0165150_g1 (Scnn1b), Mm01163272_m1 (Scnn1g), and Mn00613220_g1 (STG). Quantitative RT-PCR (qRT-PC) amplification of Gapdh was used as a control for normalization. No signal was observed in the minus RT control. Quantification was performed using the Comparative CT method: CT values were averaged from each triplet; differences between the mean CT values of the investigated genes and Gapdh were calculated as ΔCT Scnn1=CT Scnn1−CT Gapdh for normalization; and finally, Scnn1 and STG mRNA amounts relative to Gapdh were determined as 2^{ΔCT Scnn1}.

Cloning of STG Promoter Region.

Sets of primers (F:AGCACAGCCAATTTCAGCTT (SEQ ID NO: 9), R: CAGGCACAGACAGATCAGGA (SEQ ID NO: 10); F:CGTGCTAATCCCTGGAATGT (SEQ ID NO: 11), R:AACTGGGTGTTCCTCACCTG (SEQ ID NO: 12); F: CCCAAGGAGCTAAAGGGAAC (SEQ ID NO: 13), R:CAGGCACAGACAGATCAGGA (SEQ ID NO: 14); F:TGTATGCAGCCATTTCATTTTT (SEQ ID NO: 15), R:CAGGCACAGACAGATCAGGA (SEQ ID NO: 16); F:TTGTATGCAGCCATTTCATTTT (SEQ ID NO: 17), R:CTTGCTCAGCTCCTGCTTCT (SEQ ID NO: 18); F:AGGTTGAGCTTTGCTTTCCA (SEQ ID NO: 19); R:CTTGCTCAGCTCCTGCTTCT (SEQ ID NO: 20); F:AGGTTGAGCTTTGCTTTCCA (SEQ ID NO: 21), R:ACAATGAATCTGCCCAGTCC (SEQ ID NO: 22)) were designed to generate series of partially overlapping fragments of the 5′ upstrem sequence of the murine STG gene. After the optimization of amplification conditions, a series of fragments were synthesized. As a template, C57BL/6J genomic DNA was used. The cloned DNA fragments 5′ upstream of the coding region of the mouse STG gene were reconstructed into a plasmid vector and then recombined with a reporter gene, AcGFP (Aequorea coerulescens GFP), by cloning into the pAcGFP1-1 vector (Clontech) to yield STG-pAcGFP1-1 promoter-reporter constructs.

STG-Induced Expression of GFP in Mouse Oocytes.

Plasmid DNA was purified on Qiagen columns according to manufacture's instructions. The DNA was diluted to a concentration of 10 ng/μl. Two picoliters of the DNA solution were microinjected into the oocyte. Before and after the microinjection procedure, oocytes were cultured in M16 medium at 37° C. in a humidified atmosphere of 5% CO₂ in air.

Results

### Example 1

### RT-PCR Analysis of STG Expression in Mouse Taste Cells and Oocytes

RT-PCR experiments were performed with mRNA isolated from fungiform, circumvallate, foliate papilla, brain and oocytes. Several pairs of exon-intron-spanning primers were used in independent experiments and yielded the results presented in FIG. 1. STG expression was detected in the fungiform, circumvallate and foliate papilla, and oocytes, but not in the brain.

### Example 2

### RT-PCR Analysis of STG Expression in Mouse Tissues

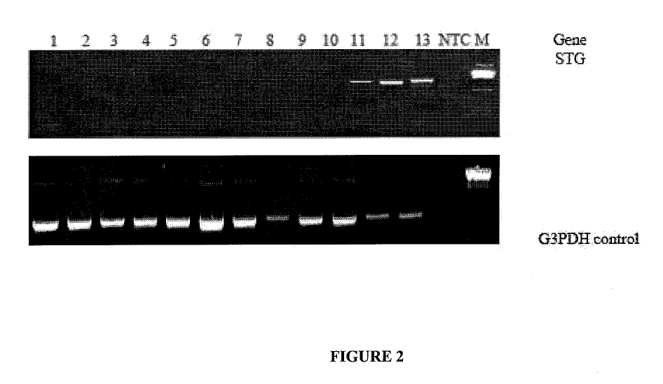

RT-PCR experiments were performed with mRNA isolated from a wide range of mouse organs and from embryos at different developmental stages. Several pairs of gene-specific primers were used in independent experiments and yielded the results presented in FIG. 2. Among the different tissues tested, STG expression was detected only in the circumvallate, fungiform and foliate papilla, and in oocytes.

### Example 3

### Quantitative RT-PCR Analysis of STG Expression in Mouse Taste Tissues

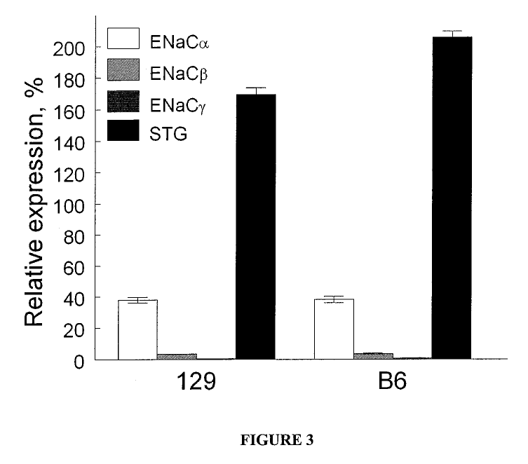

STG expression in mouse taste cells was quantified. Quantitative analyses (using normalization relative to a housekeeping gene, Gapdh) have shown that transcripts of STG were more abundant compared with ENaCα (Scnn1a), ENaCβ (Scnn1b), and ENaCγ (Scnn1g). The results are illustrated in FIG. 3.

### Example 4

### Bioinformatic Analyses of Mouse Genome Sequences

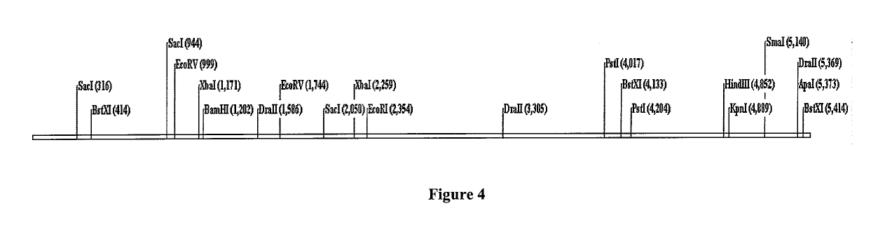

Analysis of the mouse genome sequence (illustrated in FIG. 4) has detected a region between a 5′ end of the STG gene and an adjacent gene, LOC674169. This region is likely to contain STG promoter sequences. Our gene prediction bioinformatic analyses indicate that the most likely promoter region of this gene spans ˜5.5 kb (or even a shorter) 5′ upstream sequence of the murine STG gene.

### Example 5

### Cloning the STG Promoter Region

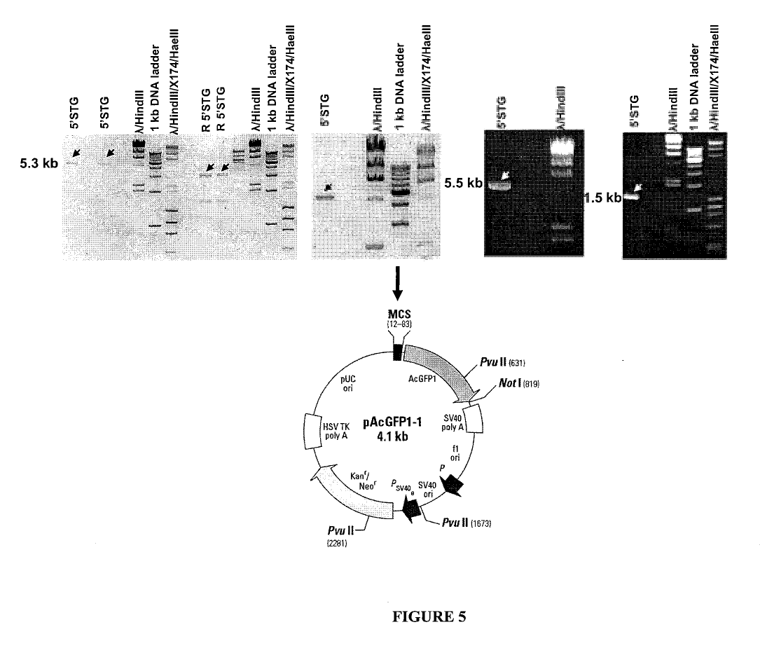

To identify the STG promoter region, fragments of the 5′ upstream region of the murine STG gene were cloned using polymerase chain reaction (PCR). As a template, C57BL/6J genomic DNA was used. Sets of primers were designed to generate series of partially overlapping fragments of the 5′ upstream sequence of the murine STG gene. After the optimization of amplification conditions, a series of fragments were synthesized (5.5 kb, 5.3 kb, 3.3 kb, 2.7 kb, 1.5 kb) (FIG. 5) which completely cover this region and are likely to contain the promoter sequences of the murine STG gene. The identity of the cloned fragments was confirmed by DNA sequencing.

The 1,179 by sequence of the promoter region of the murine STG gene immediately upstream of its start codon is (SEQ ID NO: 23):

CTGCAGCTTGTAGTTCGAGCACAGGCTCAGGATTCTAACTAG
CACATATTTGTAATTAAGAAAAAAATCCGGCAGATTGAGATT
GTTGCAGGATTTGAGAAAGGTTAATAAAACTATGGAGCTTGT
AGGGGCATTGCAGTCTGGCCTGCCTACTCCACACAGAATTAT
GATGGGTTTGGAGGATTGTTCTTTATTCCTTTGCATCCTGAT
GATGTAAAGGGTTTGCTTTTAGTTAGTTTGTAGTTTTAAAGA
GCCCATGAAGCGATGTCATTGGAAAGTTTTGCCTCAAGGAAT
GACTAATAGCCATCCATACTTTATGTAAAAAATTTTTGTCGC
TGCTTCAATACAAGAAGTTAGGACTTGGGAATTTTTCGGTGT
ATAGTATTCATATGTAGATGGTATTTTATTAGCTGATCCCTC
TGATGGAATTTTACTACAAGGCTTTGCTCTTATACAATGAGC
TTTAACATTTTGGAGTAGTTGTTGCTCCAGAAAAGATTCAAG
GGCAAATTCTTTTTTTCAATATTTGGAACATAAGTTACAGCC
TGAACAAATTGCAGCACAGAAAATTCAAGTAAGAAAAAATAA
TTTAGTTACTCTAAATGATTTTCGAAGGTTGTTAAGAGACAT
TAATCAGCTAAGACCTCATCTTAAGCTTACTACAGGAGAACT
TAAACCTCTGCTTGGTACCCTCAAGGGGGATGCAAGTCCTAA
GTCCCCTCTGACAATGAACTGATGAGGGACGAATAGCTGACT
AGCTTCCCAGAAAGTGGAGGAGGCTATTAGTCAATGACAGAT
ACATTATATAGGCTATGATCAATTGATACATTATATAGACTA
TGATCATTTGATACATTATATAGACTATGATCAATTGTTGGC
TGTTTGCATGCCAACAATTGATCATAGTCTATATAATGGTAG
AGCATTATAGAGCACCCGGGTGCTCTATAATGGTAGAGCACC
TGGGTGAGGAAGGGGCAGGGAGTACCAGGAAGTAGCCAGGTG
AGGAACACCCAGTTCCTCAGGAACTTTCAAGTGACACCTGTT
GCCACAGACCGAATCAAGAATGAGGGTGGACTGGGCAGATTC
ATTGTCTCTATAACCACTGTTCCCAGACTGTTTCTGCTGTCC
TCCACCCGACCAACAGGATAAAGGCTGCTCTCTTGGGGCCCA
GGC

### Example 6

### Construction of an STG-GFP Expression Vector

The cloned 1,179 by DNA fragment immediately 5′ upstream of the coding region of the mouse STG gene were reconstructed into a plasmid vector and then recombined with a reporter gene, AcGFP (Aequorea coerulescens GFP), by cloning into the pAcGFP1-1 vector (Clontech) to yield STG-pAcGFP1-1 promoter-reporter constructs.

### Example 7

### STG-Induced Expression of GFP in Mouse Oocytes

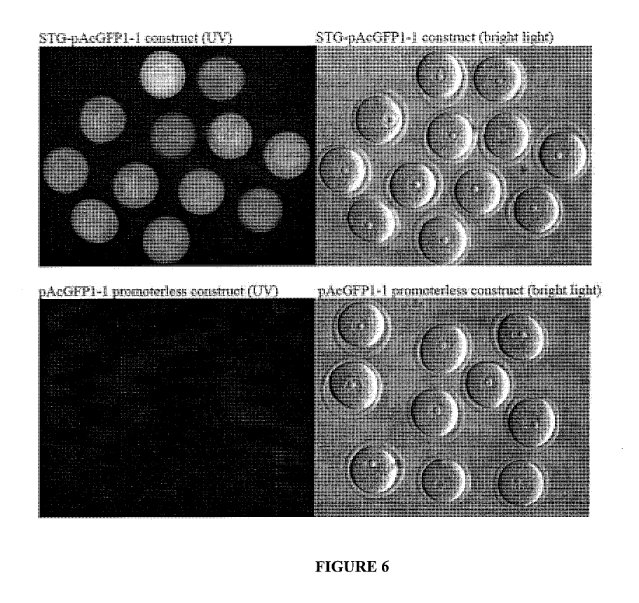

This experiment was conducted to test functionality of genomic fragments that are located upstream of the STG coding region and are expected to contain promoter sequences. The ability of these fragments to drive expression of a reporter gene (green fluorescent protein, GFP) in vitro was examined. To achieve this, mouse oocytes were injected with the plasmid construct, which has this region combined with a reporter gene (AcGFP). Because mouse oocytes endogenously express STG, injection of the STG-pAcGFP1 constructs with functional STG promoter sequences should result in GFP expression. Fluorescent signal was detected, indicating GFP expression under the STG promoter (FIG. 6).

What is claimed:

1. A vector comprising the Simian taste bud-specific gene (STG) promoter operatively linked to a reporter gene.

2. The vector of claim 1 wherein the STG promoter is the murine ortholog of the STG promoter.

3. The vector of claim 1 wherein the STG promoter is the human ortholog of the STG promoter.

4. The vector of claim 1 further comprising a polyadenylation signal downstream of said reporter gene.

5. The vector of claim 1 wherein said reporter gene is green fluorescent protein.

6. A host cell comprising the vector of claim 1.

7. The host cell according to claim 6 wherein said host cell is a mammalian cell.

8. A method for producing a cell that expresses a vector comprising the STG promoter operatively linked to a reporter gene, said method comprising the steps of:
introducing into a cell in vitro the vector of claim 1;
culturing said cell comprising said vector under conditions that allow for expression of said reporter gene;
selecting for a clone that expresses said reporter gene,
thereby producing said cell that expresses a vector comprising the STG promoter operatively linked to said reporter gene.

9. The method of claim 8 further comprising expanding said selected clone.

10. A method for differentiating between taste cells and non-taste cells in a primary taste tissue culture comprising:
introducing into cells of said primary taste tissue culture the vector of claim 1;
culturing said cells under conditions that allow for expression of said reporter gene; and
detecting expression of said reporter gene,
wherein cells that express said reporter gene are taste cells.

11. A method for identifying a test compound that stimulates or inhibits taste cells, said method comprising:
introducing into cells of a primary taste tissue culture the vector of claim 1;
culturing said cells under conditions that allow for expression of said reporter gene;
detecting expression of said reporter gene, wherein cells that express said reporter gene are taste cells;
exposing a cell that expresses said reporter gene to said test compound; and
detecting a response of said cell that expresses said reporter gene to said test compound.

12. A method for identifying a test compound that stimulates or inhibits expression of a STG promoter comprising the steps of:
introducing into a cell in vitro the vector of claim 1;
culturing said cell comprising said vector under conditions that allow for expression of said reporter gene;
selecting for a clone that expresses said reporter gene,
thereby producing said cell that expresses a vector comprising the STG promoter operatively linked to said reporter gene;
exposing said clone that expresses said reporter gene to a test compound; and
measuring the expression of said reporter gene,
wherein an increase in expression of said reporter gene in response to the test compound relative to the expression of said reporter gene in the absence of said test compound is indicative of a stimulant of said STG promoter and a decrease in expression of said reporter gene in response to the test compound relative to the expression of said reporter gene in the absence of said test compound is indicative of an inhibitor of said STG promoter.

13. A transgenic non-human mammal comprising a polynucleotide comprising the STG promoter operatively linked to a reporter gene.

14. A method for differentiating between taste cells and non-taste cells in a primary taste tissue culture comprising:
obtaining a primary taste tissue culture of the mammal of claim 13;
culturing cells of said primary taste tissue culture under conditions that allow for expression of said reporter gene; and
detecting expression of said reporter gene,
wherein cells that express said reporter gene are taste cells.

15. A method for analyzing a molecular, biochemical, or physiological property of a taste cell comprising:
obtaining a primary taste tissue culture of the mammal of claim 13;
culturing cells of said primary taste tissue culture under conditions that allow for expression of said reporter gene;
detecting expression of said reporter gene, wherein cells that express said reporter gene are taste cells;
and analyzing said molecular, biochemical, or physiological property of a cell that expresses said reporter gene.

16. A method for producing cultured taste cells comprising:
obtaining a primary taste tissue culture of the mammal of claim 13;
culturing cells of said primary taste tissue culture under conditions that allow for expression of said reporter gene;
detecting expression of said reporter gene, wherein cells that express said reporter gene are taste cells;
selecting said cells that express said reporter gene for further culture.

17. A method for identifying a test compound that stimulates or inhibits taste cells, said method comprising:
obtaining a primary taste tissue culture of the mammal of claim 13;
culturing cells of said primary taste tissue culture under conditions that allow for expression of said reporter gene;
detecting expression of said reporter gene, wherein cells that express said reporter gene are taste cells;
exposing a cell that expresses said reporter gene to said test compound; and
detecting a response of said cell that expresses said reporter gene to said test compound.

18. A method for producing a transgenic non-human mammal comprising a polynucleotide comprising the STG promoter operatively linked to a reporter gene, comprising the steps of:
introducing a transgene into a zygote of a non-human mammal, said transgene comprising a polynucleotide comprising the STG promoter operatively linked to a reporter gene;
transplanting said zygote into a pseudopregnant mouse;
allowing said zygote to develop to term; and
identifying at least one transgenic offspring containing said transgene.

19. A method for screening agents for their effect on the STG promoter comprising the steps of:
selecting taste cells from the transgenic non-human mammal according to the method of claim 16;
culturing said taste cells under conditions that allow for the expression of said reporter gene;
detecting expression of said reporter gene, wherein cells that express said reporter gene are taste cells;
exposing a cell that expresses said reporter gene to said test compound; and
detecting a response of said cell that expresses said reporter gene to said compound.

20. A method for visualizing oocytes of a mammal, said method comprising the steps of:
obtaining oocytes from a female transgenic non-human mammal of claim 13;
culturing said oocytes under conditions that allow for the expression of said reporter gene; and
detecting oocytes that express said reporter gene.

21. A method for analyzing a molecular, biochemical, or physiological property of an oocyte comprising:
obtaining oocytes from a female transgenic non-human mammal of claim 13;
culturing said oocytes under conditions that allow for expression of said reporter gene;
and analyzing said molecular, biochemical, or physiological property of an oocyte that expresses said reporter gene.

22. A method for analyzing oocyte development and fertilization comprising the steps of:
detecting expression of said reporter gene in an oocyte of a female transgenic non-human mammal of claim 13;
mating said female transgenic non-human mammal with a wild-type male non-human mammal, thereby generating a pregnant female transgenic non-human mammal; and
analyzing oocyte development, fertilization and embryonic development in said pregnant female transgenic non-human mammal.

23. A method for analyzing oocyte development and fertilization comprising the steps of:
detecting expression of said reporter gene in an oocyte of a female transgenic non-human mammal of claim 13;
fertilizing said oocyte of a female transgenic non-human mammal with sperm from a wild-type male non-human mammal in vitro, thereby generating a transgenic non-human mammal embryo;
analyzing oocyte development and fertilization of said oocyte of a female transgenic non-human mammal in vitro;
and analyzing embryonic development in said transgenic non-human mammal embryo in vitro.

24. A vector comprising the STG promoter operatively linked to a cre-recombinase gene.

25. A transgenic non-human mammal comprising a vector according to claim 24.

26. The transgenic non-human mammal of claim 25 further comprising a floxed allele of another gene.

27. A method for generating a non-human mammal comprising a DNA deletion, said method comprising mating a transgenic non-human mammal of claim 25 with a non-human mammal comprising a floxed allele, wherein the offspring of said mating comprises said DNA deletion.

28. The non-human mammal generated according to the method of claim 27.

29. A method for analyzing the effects of inactivation of a gene comprising the steps of:
obtaining a primary taste tissue culture of the non-human mammal of claim 28;
analyzing the molecular, biochemical or physiological property of a cell from said primary taste tissue culture; and
comparing said molecular, biochemical or physiological property of a cell from said primary taste culture of the non-human mammal to said molecular, biochemical or physiological property of a cell from a primary taste culture of a wild-type mammal.

30. A method for analyzing the effects of inactivation of the gene comprising the steps of:
obtaining an oocyte of a female non-human mammal of claim 28;
analyzing the molecular, biochemical or physiological property of said oocyte; and
comparing said molecular, biochemical or physiological property of said oocyte to said molecular, biochemical or physiological property of an oocyte of a wild-type mammal.


SEQUENCE LISTING (23 sequences)

SEQ ID NO: 1 - DNA, 20 bp, Artificial Sequence
  Synthetic primer
ttccttcaccagcctcctta

SEQ ID NO: 2 - DNA, 20 bp, Artificial Sequence
  Synthetic primer
gctgcaaaccagttgtgaga

SEQ ID NO: 3 - DNA, 20 bp, Artificial Sequence
  Synthetic primer
gctgcaaaccagttgtgaga

SEQ ID NO: 4 - DNA, 20 bp, Artificial Sequence
  Synthetic primer
ttccttcaccagcctcctta

SEQ ID NO: 5 - DNA, 22 bp, Artificial Sequence
  Synthetic primer
tgcagagccacgcaggtgggag

SEQ ID NO: 6 - DNA, 21 bp, Artificial Sequence
  Synthetic primer
cgaccaacaggataaaggctg

SEQ ID NO: 7 - DNA, 20 bp, Artificial Sequence
  Synthetic primer
gtactggggtccttgggaac

SEQ ID NO: 8 - DNA, 20 bp, Artificial Sequence
  Synthetic primer
cccgctactcttttgactcg

SEQ ID NO: 9 - DNA, 20 bp, Artificial Sequence
  Synthetic primer
agcacagccaatttcagctt

SEQ ID NO: 10 - DNA, 20 bp, Artificial Sequence
  Synthetic primer
caggcacagacagatcagga

SEQ ID NO: 11 - DNA, 20 bp, Artificial Sequence
  Synthetic primer
cgtgctaatccctggaatgt

SEQ ID NO: 12 - DNA, 20 bp, Artificial Sequence
  Synthetic primer
aactgggtgttcctcacctg

SEQ ID NO: 13 - DNA, 20 bp, Artificial Sequence
  Synthetic primer
cccaaggagctaaagggaac

SEQ ID NO: 14 - DNA, 20 bp, Artificial Sequence
  Synthetic primer
caggcacagacagatcagga

SEQ ID NO: 15 - DNA, 22 bp, Artificial Sequence
  Synthetic primer
tgtatgcagccatttcattttt

SEQ ID NO: 16 - DNA, 20 bp, Artificial Sequence
  Synthetic primer
caggcacagacagatcagga

SEQ ID NO: 17 - DNA, 22 bp, Artificial Sequence
  Synthetic primer
ttgtatgcagccatttcatttt

SEQ ID NO: 18 - DNA, 20 bp, Artificial Sequence
  Synthetic primer
cttgctcagctcctgcttct

SEQ ID NO: 19 - DNA, 20 bp, Artificial Sequence
  Synthetic primer
aggttgagctttgctttcca

SEQ ID NO: 20 - DNA, 20 bp, Artificial Sequence
  Synthetic primer
cttgctcagctcctgcttct

SEQ ID NO: 21 - DNA, 20 bp, Artificial Sequence
  Synthetic primer
aggttgagctttgctttcca

SEQ ID NO: 22 - DNA, 20 bp, Artificial Sequence
  Synthetic primer
acaatgaatctgcccagtcc

SEQ ID NO: 23 - DNA, 1179 bp, Mouse sp.
  misc_feature: Promoter region of the murine STG gene immediately upstream of its start codon
ctgcagcttgtagttcgagcacaggctcaggattctaactagcacatatttgtaattaagaaaaaaatccggcagattgagattgttgcaggatttgagaaaggttaataaaactatggagcttgtaggggcattgcagtctggcctgcctactccacacagaattatgatgggtttggaggattgttctttattcctttgcatcctgatgatgtaaagggtttgcttttagttagtttgtagttttaaagagcccatgaagcgatgtcattggaaagttttgcctcaaggaatgactaatagccatccatactttatgtaaaaaatttttgtcgctgcttcaatacaagaagttaggacttgggaatttttcggtgtatagtattcatatgtagatggtattttattagctgatccctctgatggaattttactacaaggctttgctcttatacaatgagctttaacattttggagtagttgttgctccagaaaagattcaagggcaaattctttttttcaatatttggaacataagttacagcctgaacaaattgcagcacagaaaattcaagtaagaaaaaataatttagttactctaaatgattttcgaaggttgttaagagacattaatcagctaagacctcatcttaagcttactacaggagaacttaaacctctgcttggtaccctcaagggggatgcaagtcctaagtcccctctgacaatgaactgatgagggacgaatagctgactagcttcccagaaagtggaggaggctattagtcaatgacagatacattatataggctatgatcaattgatacattatatagactatgatcatttgatacattatatagactatgatcaattgttggctgtttgcatgccaacaattgatcatagtctatataatggtagagcattatagagcacccgggtgctctataatggtagagcacctgggtgaggaaggggcagggagtaccaggaagtagccaggtgaggaacacccagttcctcaggaactttcaagtgacacctgttgccacagaccgaatcaagaatgagggtggactgggcagattcattgtctctataaccactgttcccagactgtttctgctgtcctccacccgaccaacaggataaaggctgctctcttggggcccaggc